(19) United States
(12) Patent Application Publication
(10) Pub. No.: US 2025/0349403 A1
(43) Pub. Date: Nov. 13, 2025

(54) METHOD FOR PROVIDING BIOLOGICAL INFORMATION AND ELECTRONIC DEVICE SUPPORTING SAME

(71) Applicant: Samsung Electronics Co., Ltd., Suwon-si (KR)

(72) Inventors: Hyeeun CHOI, Suwon-si (KR); Heummo GU, Suwon-si (KR); Eungsun KIM, Suwon-si (KR); Seyoun OH, Suwon-si (KR)

(21) Appl. No.: 19/276,743

(22) Filed: Jul. 22, 2025

Related U.S. Application Data
(63) Continuation of application No. PCT/KR2024/001259, filed on Jan. 26, 2024.

(30) Foreign Application Priority Data
Feb. 27, 2023 (KR) 10-2023-0026019
Mar. 13, 2023 (KR) 10-2023-0032370

(51) Int. Cl. G16H 10/60; G16H 40/63

(52) U.S. Cl. CPC G16H 10/60; G16H 40/63

(57) ABSTRACT

An electronic device is provided including a first and second sensors, memory storing instructions, and at least one processor. The instructions, when executed by the at least one processor, cause the electronic device to, based on identifying that a user is in a sleep state, detect a movement of the electronic device via the first sensor, based on a magnitude of the movement being less than or equal to a threshold, acquire, based on a biosignal acquired via the second sensor, biometric information of the user, acquire a time period corresponding to a designated sleep stage among sleep stages of the sleep state, and identifying, among the acquired biometric information, biometric information acquired during a third time period, the third time period being an overlapping time period between a first time period during which the biometric information is acquired and a second time period corresponding to the designated sleep stage.

### CROSS-REFERENCE TO RELATED APPLICATION(S)

This application is a continuation application, claiming priority under 35 U.S.C. § 365(c), of an International application No. PCT/KR2024/001259, filed on Jan. 26, 2024, which is based on and claims the benefit of a Korean patent application number 10-2023-0026019, filed on Feb. 27, 2023, in the Korean Intellectual Property Office, and of a Korean patent application number 10-2023-0032370, filed on Mar. 13, 2023, in the Korean Intellectual Property Office, the disclosure of each of which is incorporated by reference herein in its entirety.

### BACKGROUND

### 1. Field

The disclosure relates to a method for providing biometric information and an electronic device supporting the same.

### 2. Description of Related Art

Electronic devices have been evolving in various forms to enhance user convenience and are becoming smaller in size to allow for easy portability by users. In recent years, with growing interest in personal health, electronic devices have been used to measure biosignals associated with the human body and to provide biometric information based on the measured biosignals. For example, an electronic device (e.g., a wearable device) may acquire a photoplethysmogram (PPG) signal through an optical sensor (e.g., a PPG sensor) for acquiring a biosignal and may provide blood pressure, based on the acquired PPG signal.

A user's blood pressure may be associated with various diseases. For example, by comparing blood pressure measured while the user is awake (hereinafter referred to as “daytime blood pressure”) with blood pressure measured while the user is asleep (hereinafter referred to as “nighttime blood pressure”), a blood pressure pattern of the user may be identified. The pattern may correspond to one of a dipper pattern (e.g., nighttime blood pressure decreases by approximately 10% or more compared to daytime blood pressure), a non-dipper pattern (e.g., nighttime blood pressure decreases by less than approximately 10% compared to daytime blood pressure), an extreme dipper pattern (e.g., nighttime blood pressure decreases by approximately 20% or more compared to daytime blood pressure), or a riser pattern (e.g., nighttime blood pressure increases compared to daytime blood pressure). Based on the identified blood pressure pattern, the likelihood of occurrence of various diseases, such as heart disease, stroke, heart failure, or retinal disorders, may be predicted.

The above information is presented as background information only to assist with an understanding of the disclosure. No determination has been made, and no assertion is made, as to whether any of the above might be applicable as prior art with regard to the disclosure.

### SUMMARY

Daytime blood pressure may be measured while the user is in a physically and mentally stable condition (hereinafter referred to as a “resting state”), whereas nighttime blood pressure may be difficult to measure in such a resting state. For example, when a cuff-type blood pressure measuring device is used to measure a user's blood pressure while the user is asleep, periodic pressure and noise generated during the blood pressure measurement may disturb the user's sleep. As a result, it may be difficult for the user to consciously maintain a resting state, thereby making it difficult to acquire blood pressure that reflects the user's resting state. For example, when the user's blood pressure is measured periodically while the user is asleep, the blood pressure may be measured even when the user is not in a resting state or is not in a true sleep state. Moreover, when using an electronic device capable of acquiring biometric information (e.g., a wearable device) to measure blood pressure during a user's sleep, the blood pressure may be measured even when the user is in motion and/or not mentally stable during sleep. In addition, when the electronic device continuously measures blood pressure while the user is asleep, power consumption by the electronic device may increase.

Aspects of the disclosure of the disclosure are to address at least the above-mentioned problems and/or disadvantages and to provide at least the advantages described below. Accordingly, an aspect of the disclosure is to provide a method for providing biometric information and an electronic device supporting the same, wherein accurate biometric information (e.g., nighttime blood pressure) can be acquired based on the movement and/or the sleep stage detected by the electronic device.

Additional aspects will be set forth in part in the description which follows and, in part, will be apparent from the following description, or may be learned by practice of the presented embodiments.

In accordance with an aspect of the disclosure, an electronic device is provided. The electronic device includes a first sensor configured to detect a movement of the electronic device, a second sensor configured to measure a biosignal of a user wearing the electronic device, memory storing instructions, and at least one processor operably connected to the first sensor, the second sensor, and the memory. The instructions, when executed by the at least one processor individually or collectively, cause the electronic device to, based on identifying that the user is in a sleep state, detect the movement of the electronic device via the first sensor, based on a magnitude of the movement of the electronic device being less than or equal to a threshold magnitude, acquire, based on the biosignal acquired via the second senso, biometric information of the user, acquire a time period corresponding to a designated sleep stage among sleep stages of the sleep state, and identify, among the acquired biometric information, biometric information acquired during a third time period, the third time period being an overlapping time period between a first time period during which the biometric information is acquired and a second time period corresponding to the designated sleep stage.

In accordance with another aspect of the disclosure, a method performed by an electronic device is provided. The method includes, based on identifying that a user wearing the electronic device is in a sleep state, detecting a movement of the electronic device via a first sensor of the electronic device, based on a magnitude of the movement of the electronic device being less than or equal to a threshold magnitude, acquiring, based on a biosignal of the user acquired via a second sensor of the electronic device, biometric information of the user, acquiring a time period corresponding to a designated sleep stage among sleep stages of the sleep state, and identifying, among the acquired biometric information, biometric information acquired during a third time period, the third time period being an overlapping time period between a first time period during which the biometric information is acquired and a second time period corresponding to the designated sleep stage.

In accordance with another aspect of the disclosure, an electronic device is provided. The electronic device includes a sensor configured to measure a biosignal of a user wearing the electronic device, memory storing instructions, and at least one processor including processing circuitry. The instructions, when executed by the at least one processor individually or collectively, cause the electronic device to, based on identification that the user is in a sleep state, acquire a sleep stage of the sleep state, acquire the biosignal via the sensor, based on identification that the sleep stage is a designated sleep stage, and acquire biometric information of the user based on the biosignal.

In accordance with another aspect of the disclosure, one or more non-transitory computer-readable storage media storing instructions that, when executed by at least one processor of an electronic device individually or collectively, cause the electronic device to perform operations, is provided, The operations include, based on identifying that a user wearing the electronic device is in a sleep state, detecting a movement of the electronic device via a first sensor of the electronic device, based on a magnitude of the movement of the electronic device being less than or equal to a threshold magnitude, acquiring, based on a biosignal of the user acquired via a second sensor of the electronic device, biometric information of the user, acquiring a time period corresponding to a designated sleep stage among sleep stages of the sleep state, and identifying, among the acquired biometric information, biometric information acquired during a third time period, the third time period being an overlapping time period between a first time period during which the biometric information is acquired and a second time period corresponding to the designated sleep stage.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of an electronic device in a network environment according to an embodiment of the disclosure;

FIG. 2A is a front perspective view of an electronic device according to an embodiment of the disclosure;

FIG. 2B is a rear perspective view of the electronic device of FIG. 2A according to an embodiment of the disclosure;

FIG. 2C is an exploded perspective view of the electronic device of FIG. 2A according to an embodiment of the disclosure;

FIG. 3 is a block diagram of an electronic device according to an embodiment of the disclosure;

FIG. 4 is a flowchart for illustrating a method for providing biometric information according to an embodiment of the disclosure;

FIG. 5 is a diagram for illustrating sleep stages according to an embodiment of the disclosure;

FIG. 6 is a flowchart for illustrating a method for configuring a detection time point for a movement of an electronic device, based on the magnitude of the movement of the electronic device according to an embodiment of the disclosure;

FIG. 7 is a diagram for illustrating a method for configuring a detection time point for a movement of an electronic device, based on the magnitude of the movement of the electronic device according to an embodiment of the disclosure;

FIG. 8 is a diagram for illustrating a method for configuring an interval for detecting a movement of an electronic device, based on the elapsed time from the time point at which sleep begins, according to an embodiment of the disclosure;

FIG. 9 is a flowchart for illustrating a method for determining an initial time point for detecting a movement of an electronic device, based on a user's sleep history, according to an embodiment of the disclosure;

FIG. 10 is a flowchart for illustrating a method for providing biometric information according to an embodiment of the disclosure;

FIG. 11 illustrates a method for providing biometric information according to an embodiment of the disclosure;

FIG. 12 is a diagram for illustrating a method for providing biometric information according to an embodiment of the disclosure; and

FIG. 13 is a flowchart for illustrating a method for providing biometric information according to an embodiment of the disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a wireless fidelity (Wi-Fi) chip, a Bluetooth® chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

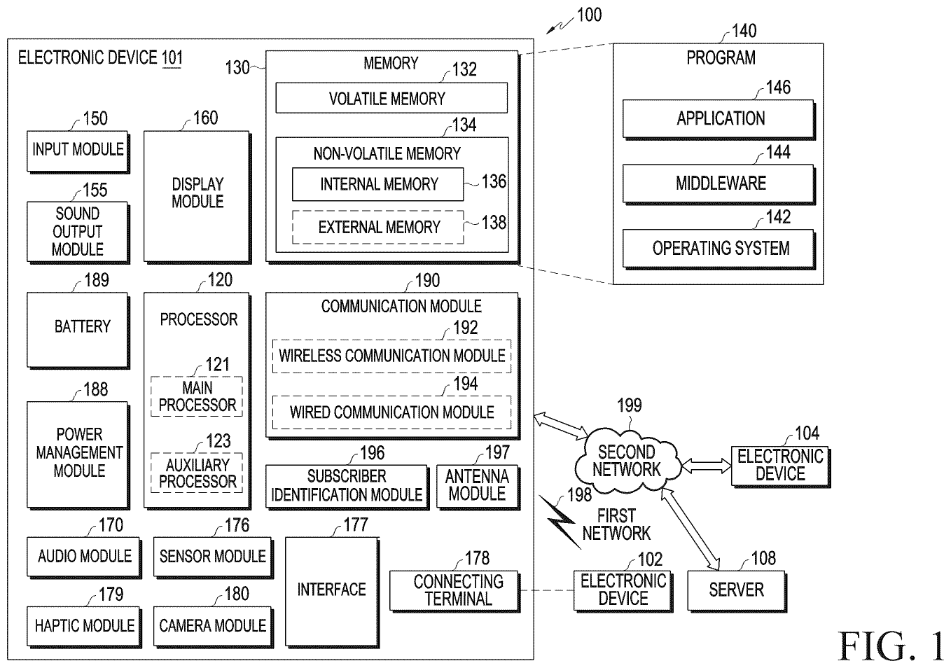

FIG. 1 is a block diagram illustrating an electronic device 101 in a network environment 100 according to an embodiment of the disclosure.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a fifth generation (5G) network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a fourth generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the millimeter wave (mmWave) band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

The electronic device according to an embodiment may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that an embodiment of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with an embodiment of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

An embodiment as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to an embodiment of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to an embodiment, each component (e.g., a module or a program) of the above-described components may include a single entity or multiple entities, and some of the multiple entities may be separately disposed in different components. According to an embodiment, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to an embodiment, operations performed by the module, the program, or another component may be carried out sequentially, in parallel, repeatedly, or heuristically, or one or more of the operations may be executed in a different order or omitted, or one or more other operations may be added.

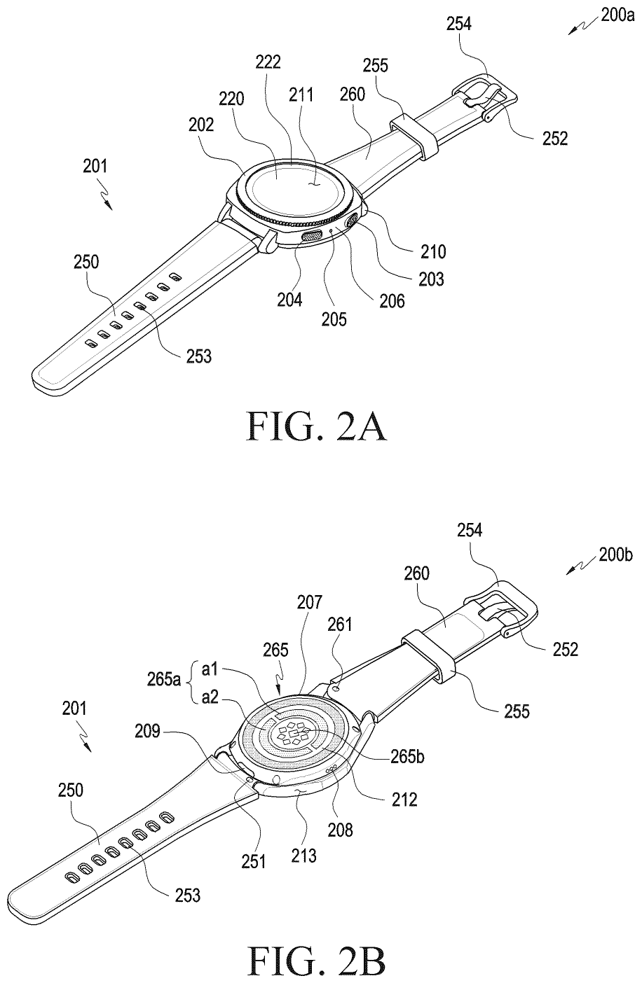

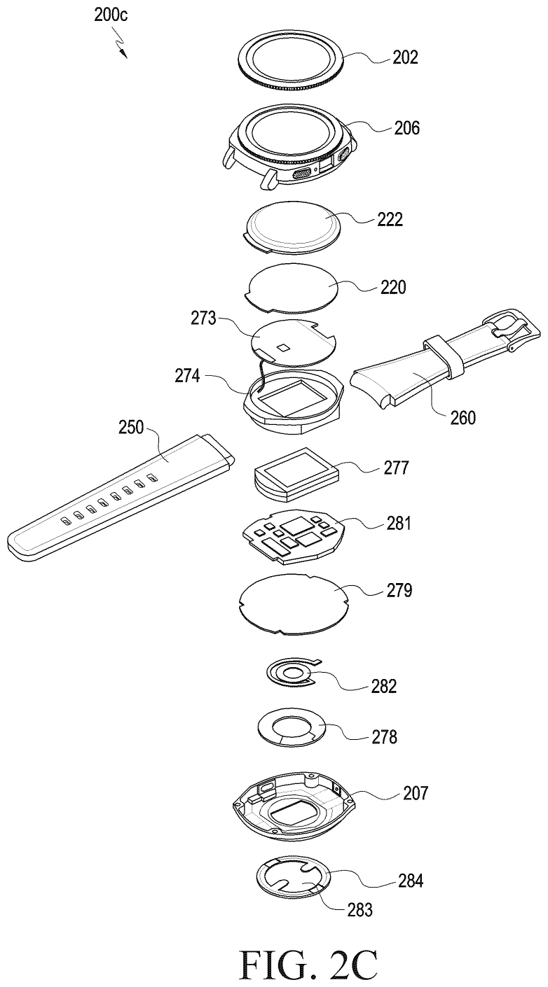

FIG. 2A is a front perspective view 200a of an electronic device 201 according to an embodiment of the disclosure.

FIG. 2B is a rear perspective view 200b of the electronic device 201 of FIG. 2A according to an embodiment of the disclosure.

Referring to FIGS. 2A and 2B, the electronic device 201 (e.g., the electronic device 101 of FIG. 1) according to an embodiment may include a housing 210 including a first surface (or front surface) 211, a second surface (or rear surface) 212, and a lateral surface 213 surrounding a space between the first surface 211 and the second surface 212, and wearing members 250, 260 connected to at least a part of the housing 210 and configured to detachably attach the electronic device 201 to a part (e.g., a wrist or an ankle) of the user's body. In another embodiment (not shown), the housing 210 may refer to a structure configuring at least a part of one or more of the first surface 211, the second surface 212, and the lateral surface 213, which are illustrated in FIGS. 2A and 2B. In an embodiment, the first surface 211 may be configured by a front plate 222 that is at least partially transparent (e.g., a glass plate including various coating layers, or a polymer plate). The second surface 212 may be configured by a rear plate 207 that is substantially opaque. In some embodiments, when the electronic device 201 includes a sensor module 265 (e.g., the sensor module 176 of FIG. 1) disposed on the second surface 212 thereof, the rear plate 207 may include at least a partially transparent area.

The rear plate 207 may be made of, for example, coated or colored glass, ceramic, polymer, metal (e.g., aluminum, stainless steel (STS), or magnesium), or a combination of at least two of the materials above. The lateral surface 213 may be configured by a lateral bezel (or “lateral member”) 206 that is coupled with the front plate 222 and the rear plate 207 and includes a metal and/or polymer. In some embodiments, the rear plate 207 and the lateral bezel structure 206 may be formed integrally and include the same material (e.g., a metal material such as aluminum). The wearing members 250, 260 may be made of various materials and be configured in various shapes. The integral and multiple unit links may be configured using woven fabric, leather, rubber, urethane, metal, ceramic, or a combination of at least two of the materials to be movable relative to each other.

According to an embodiment, the electronic device 201 may include at least one of a display 220 (see FIG. 2C) (e.g., the display module 160 of FIG. 1), an audio module 205, 208 (e.g., the audio module 170 of FIG. 1), a sensor module 265 (e.g., the sensor module 176 of FIG. 1), a key input device 202, 203, 204 (e.g., the input module 150 of FIG. 1), and a connector hole 209. In some embodiments, at least one of the components (e.g., the key input device 202, 203, 204, the connector hole 209, or the sensor module 265) may be omitted from the electronic device 201 or another component may be added to the electronic device 201.

According to an embodiment, the electronic device 201 may include a plurality of electrodes for measuring a biosignal, and at least one of the plurality of electrodes may be disposed at one or more of the position of the key input device 202, 203, or 204, the position of the lateral bezel structure 206, the position of the display 220, or the position of the housing 210. The wheel key 202 among the key input devices may include a rotary bezel.

The display 220 may be exposed through, for example, a substantial portion of the front plate 222. The shape of the display 220 may correspond to the shape of the front plate 222, and may have various shapes such as a circle, an oval, or a polygon. The display 220 may be coupled with or positioned adjacent to a touch detection circuit, a pressure sensor capable of measuring the intensity (pressure) of a touch, and/or a fingerprint sensor.

According to an embodiment, the display 220 may include at least one transparent electrode for measuring a biosignal, among the plurality of electrodes for measuring a biosignal.

The audio module 205, 208 may include a microphone hole 205 and a speaker hole 208. A microphone 205 may be placed inside the microphone hole 205 to capture external sound, and in some embodiments, multiple microphones may be arranged to detect the direction of sound. The speaker hole 208 may be used for an external speaker or a receiver for voice calls. In some embodiments, a speaker may be included without a speaker hole (e.g., a piezoelectric speaker).

The sensor module 265 may generate electrical signals or data values corresponding to the internal operating state of the electronic device 201 or the external environmental conditions. The sensor module 265, which is a biometric sensor module 265 (e.g., HRM sensor) disposed on the second surface 212 of the housing 210, may include, for example, an ECG sensor 265a including at least two electrodes a1, a2 for electrocardiogram measurement, and a PPG sensor 265b for heart rate measurement. The electronic device 201 may further include at least one of sensor modules not shown herein, for example, a gesture sensor, a gyro sensor, a barometric pressure sensor, a magnetic sensor, an acceleration sensor, a grip sensor, a color sensor, an infrared (IR) sensor, a biometric sensor, a temperature sensor, a humidity sensor, or an illuminance sensor.

The key input devices 202, 203, 204 may include a wheel key 202 disposed on the first surface 211 of the housing 210 and rotatable in at least one direction, and/or a side key button 203, 204 disposed on the lateral surface 213 of the housing 210. The wheel key 202 may have a shape corresponding to the shape of the front plate 222. In other embodiments, one of the key input devices 202, 203, 204 may be implemented, on the display 220, in another shape, such as a soft key. The connector hole 209 may accommodate a connector (e.g., a USB connector) for transmitting and/or receiving power and data to and/or from an external electronic device, and may further include another connector hole (not shown) for accommodating a connector for transmitting and receiving audio signals to and from an external electronic device. The electronic device 201 may further include, for example, a connector cover (not shown) that at least partially covers the connector hole 209 and blocks the ingress of foreign substances into the connector hole.

The wearing member 250, 260 may be detachably attached to at least a part of the housing 210 by using a locking member 251, 261. The locking member 251, 261 may include a fastening component, such as a pogo pin, and may be replaced with a protrusion(s) or recess(es) configured in the wearing member 250, 260 according to an embodiment. For example, the wearing member 250, 260 may be coupled in a manner that engages with the recess(es) or protrusion(s) configured in the housing 210. The wearing member 250, 260 may include one or more of a fixing member 252, a fixing member fastening hole 253, a band guide member 254, and a band fixing ring 255.

The fixing member 252 may be configured to secure the housing 210 and the wearing member 250, 260 to a part (e.g., wrist, ankle) of the user's body. The fixing member fastening hole 253 may correspond to the fixing member 252 to secure the housing 210 and the wearing member 250, 260 to a part of the user's body. The band guide member 254 may be configured to limit the movement range of the fixing member 252 when the fixing member 252 is fastened to the fixing member fastening hole 253, thereby allowing the wearing member 250, 260 to be tightly attached to the user's body. The band fixing ring 255 may limit the movement range of the wearing member 250, 260 when the fixing member 252 are fastened with the fixing member fastening hole 253.

FIG. 2C is an exploded perspective view 200c illustrating the electronic device 201 of FIG. 2A according to an embodiment of the disclosure.

Referring to FIG. 2C, the electronic device 201 (e.g., the electronic device 101 of FIG. 1) may include a lateral bezel structure 206, a wheel key 202, a front plate 222, a display 220, a first antenna 273, a support member 274 (e.g., a bracket), a battery 277, a first printed circuit board 281 (e.g., a printed circuit board (PCB), a printed board assembly (PBA), a flexible PCB (FPCB), or a rigid-flexible PCB (RFPCB)), a second printed circuit board 282, a sealing member 279, a second antenna 278, a rear housing 207 and a rear cover 283, a signal detection unit 284 (e.g., the electrode of the ECG 265a or BIA sensor and the PPG sensor 265b of FIG. 2B), and a fastening member 250, 260. At least one of the components of the electronic device 201 may be the same as or similar to at least one of the components of the electronic device 201 of FIG. 2A or FIG. 2B, and redundant descriptions will be omitted hereinafter.

In an embodiment, the support member 274 may be disposed inside the electronic device 201 and connected to the lateral bezel structure 206 or may be configured integrally with the lateral bezel structure 206. The support member 274 may be made of, for example, a metal material and/or a non-metallic (e.g., a polymer) material. The support member 274 may have one surface coupled to the display 220 and the other surface coupled to the first printed circuit board 281. The printed circuit board 281 may be equipped with a processor (e.g., the processor 120 of FIG. 1), memory (e.g., the memory 130 of FIG. 1), and/or an interface (e.g., the interface 177 of FIG. 1). The processor may include, for example, one or more of a central processing unit, an application processor, a graphic processing unit (GPU), an application processor signal processing unit, or a communication processor.

In an embodiment, the memory may include volatile memory (e.g., the volatile memory 132 of FIG. 1) or non-volatile memory (e.g., the non-volatile memory 134 of FIG. 1). The interface may include, for example, a high definition multimedia interface (HDMI), a universal serial bus (USB) interface, an SD card interface, and/or an audio interface.

In an embodiment, the interface may electrically or physically connect the electronic device 201 to an external electronic device, for example, and may include a USB connector, an SD card/MMC connector, or an audio connector.

In an embodiment, the battery 277 may include, for example, a non-rechargeable primary battery, a rechargeable secondary battery, or a fuel cell, as a device for supplying power to at least one component of the electronic device 201. At least a part of the battery 277 may be disposed, for example, on substantially the same plane as the printed circuit board 281. The battery 277 may be disposed integrally within the electronic device 201, or may be configured to be attachable to and detachable from the electronic device 201.

In an embodiment, the first antenna 273 may be positioned between the display 220 and the support member 274. The first antenna 273 may include, for example, a near field communication (NFC) antenna, a wireless charging antenna, and/or a magnetic secure transmission (MST) antenna. The first antenna 273 may be configured, for example, to perform short-range communication with an external device, to wirelessly transmit and receive power for charging, and to transmit a magnetic-based signal including a short-range communication signal or payment data. In another embodiment, the antenna structure may be configured by at least a part of the lateral bezel structure 206 and/or the support member 274, or a combination thereof.

In an embodiment, the second antenna 278 may be disposed between the circuit board 281 and the rear plate 207. The second antenna 278 may include, for example, a near field communication (NFC) antenna, a wireless charging antenna, and/or a magnetic secure transmission (MST) antenna. The second antenna 278 may be configured, for example, to perform short-range communication with an external device, to wirelessly transmit and receive power for charging, or to transmit a magnetic-based signal including a short-range communication signal or payment data. In another embodiment, the antenna structure may be configured by at least a part of the lateral bezel structure 206 and/or the rear plate 207, or a combination thereof.

In an embodiment, the sealing member 279 may be positioned between the lateral bezel structure 206 and the rear plate 207. The sealing member 279 may be configured to block moisture and foreign substances from entering the space enclosed by the lateral bezel structure 206 and the rear plate 207 from the outside. In addition, the sealing member 279 may shield electromagnetic signals. For example, the sealing member 279 may perform a shielding function for shielding electromagnetic interference (EMI) or other various electrical signals.

In an embodiment, the rear housing 207 and the rear cover 283 may support various components included in the electronic device 201. The rear housing 207 and the rear cover 283 may be included, for example, in the rear plate 207 described above in FIG. 2B.

In an embodiment, the rear cover 283 may be at least partially made of a transparent material capable of transmitting light. For example, a sensor (not shown) disposed on the second printed circuit board 282 may include a light-emitting part and a light-receiving part. The light-emitting part may emit light to the outside through a part of the rear cover 283 made of a transparent material, and the light-receiving part may receive external light through another part of the rear cover 283 made of a transparent material. For instance, the sensor including the light-emitting part and the light-receiving part may be a sensor that measures blood flow using a photoplethysmography (PPG) method to acquire information related to a user's heart rate.

In an embodiment, the signal detection unit 284 may include an electrode that is in contact with the user's body (e.g., an electrode of an ECG sensor 265a or BIA sensor of FIG. 2B). For example, the signal detection unit 284 may be configured at least partially on a part of the rear cover 283 that may be in contact with the user's body.

In an embodiment, the second printed circuit board 282 may include at least one of the various components of the electronic device described above in FIG. 1. In an embodiment, the second printed circuit board 282 may be electrically connected to the first printed circuit board 281 described above. In an embodiment, internal electronic components of the electronic device may be distributed and arranged on the first printed circuit board 281 and the second printed circuit board 282. In an embodiment, the second printed circuit board 282 may be connected to the signal detection unit 284 to receive a signal detected by the signal detection unit 284 and process the signal. In some embodiments, a sensing processing circuit or microcontroller unit (MCU), which is separate from the processor that controls the overall operation of the electronic device 201, may be disposed on the second printed circuit board 282 to independently/primarily process signals detected by the signal detection unit 284 and/or a sensor (e.g., a PPG sensor), which are disposed on the second printed circuit board.

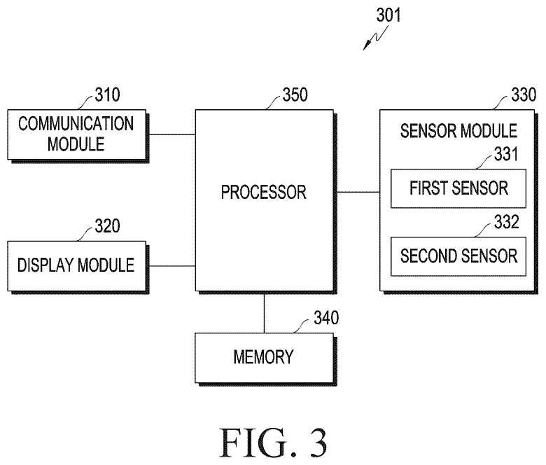

FIG. 3 is a block diagram of an electronic device 301 according to an embodiment of the disclosure.

Referring to FIG. 3, in an embodiment, the electronic device 301 may be the electronic device 101 described through FIG. 1 and/or the electronic device 201 described through FIGS. 2A, 2B, and 2C. In an embodiment, the electronic device 301 may be an electronic device including at least one of the components of the electronic device 101 of FIG. 1 and/or at least one of the components of the electronic device 201 of FIGS. 2A, 2B, and 2C.

In an embodiment, the electronic device 301 may include a communication module 310, a display module 320, a sensor module 330, memory 340, and/or a processor 350.

In an embodiment, the communication module 310 may be the communication module 190 of FIG. 1. The communication module 310 may support communication between the electronic device 301 and the external electronic device.

In an embodiment, when information related to biometric information is acquired from the electronic device 301 (e.g., a wearable device), the communication module 310 may transmit the acquired information to an external electronic device (e.g., a smartphone) (e.g., the electronic device 102, 104 of FIG. 1).

In an embodiment, when information related to the user's sleep stage is acquired from the external electronic device, the communication module 310 may receive the acquired information related to the sleep stage from the external electronic device.

In an embodiment, the communication module 310 may transmit and receive a wireless signal (e.g., a Wi-Fi signal) to and from the external electronic device, thereby allowing the electronic device 301 (or the external electronic device) to acquire information related to the user's sleep stage.

In an embodiment, the display module 320 may be included in the display module 160 of FIG. 1.

In an embodiment, the display module 320 may display information related to biometric information. The information related to biometric information displayed by the display module 320 will be described in detail later.

In an embodiment, the sensor module 330 may be the sensor module 176 of FIG. 1 or the sensor module 265 of FIG. 2B.

In an embodiment, the sensor module 330 may include a first sensor 331 and/or a second sensor 332.

In an embodiment, the first sensor 331 may sense (e.g., detect) a movement of the electronic device 301 (and a user wearing the electronic device 301). For example, the first sensor 331 may include an inertial sensor (inertial measurement unit (IMU) sensor) (referred to as a “motion sensor”) that may detect a movement of the electronic device 301. For example, the first sensor 331 may include an acceleration sensor and/or a gyro sensor. However, the first sensor 331 for detecting a movement of the electronic device 301 is not limited to the examples described above.

In an embodiment, the second sensor 332 may acquire (e.g., measure) a biosignal. For example, the second sensor 332 may include an optical sensor (e.g., the PPG sensor 265b of FIG. 2B) for measuring a biosignal (e.g., a photoplethysmogram (PPG) signal). For example, the second sensor 332 may include a sensor capable of measuring a biosignal (e.g., an electrocardiogram (ECG) signal, a galvanic skin response (GSR) signal, an electroencephalogram (EEG) signal, a bio-electrical impedance analysis (BIA) signal) by using one or more electrodes.

In the above examples, the sensor module 330 has been described as including the first sensor 331 and/or the second sensor 332, but it is not limited thereto. For example, the sensor module 330 may further include at least one sensor included in the sensor module 176 of FIG. 1 and/or at least one sensor included in the sensor module 265 of FIG. 2B.

In an embodiment, the memory 340 may be the memory 130 of FIG. 1.

In an embodiment, the memory 340 may store information for performing an operation for providing biometric information. The information stored by the memory 340 for the operation for providing biometric information will be described in detail later.

In an embodiment, the processor 350 may be the processor 120 of FIG. 1.

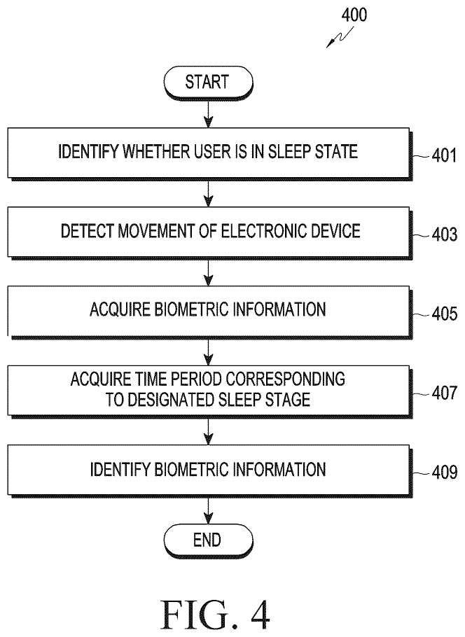

In an embodiment, the processor 350 may control the overall operation for providing biometric information. In an embodiment, the processor 350 may include one or more processors for providing biometric information. The operations performed by the processor 350 to provide biometric information will be described below with reference to FIG. 4.

The electronic device 301 is illustrated in FIG. 3 as including the communication module 310, the display module 320, the sensor module 330, the memory 340, and the processor 350, but it is not limited thereto. For example, the electronic device 301 may further include at least one component included in the electronic device 101 of FIG. 1 and/or the electronic device 201 of FIG. 2.

FIG. 4 is a flowchart 400 for illustrating a method for providing biometric information according to an embodiment of the disclosure.

In the following embodiments, the respective operations may be performed sequentially, but are not necessarily performed sequentially. For example, the order of the operations may be changed, and at least two operations may be performed in parallel.

According to an embodiment, operations 401 to 409 may be understood to be performed by a processor (e.g., the processor 350 of FIG. 3) of an electronic device (e.g., the electronic device 301 of FIG. 3).

Referring to FIG. 4, in an embodiment, in operation 401, the processor 350 may identify whether the user is in a sleep state. For example, the processor 350 may identify whether the user wearing the electronic device 301 (e.g., a wearable device) has started sleeping.

In an embodiment, the processor 350 may acquire information on the movement of the electronic device 301 (e.g., the magnitude of the movement of the electronic device 301) via the first sensor 331 (e.g., an acceleration sensor). Based on the information on the movement of the electronic device 301, the processor 350 may identify whether the magnitude of the movement of the electronic device 301 is less than or equal to a threshold magnitude for a designated time period. When the magnitude of the movement of the electronic device 301 is identified to be less than or equal to a threshold magnitude for the designated time period, the processor 350 may identify that the user has started sleeping. However, the method by which the processor 350 identifies whether the user is in a sleep state is not limited to the examples described above.

In an embodiment, in operation 403, the processor 350 may detect a movement of the electronic device 301 via the first sensor 331, based on identification that the user is in a sleep state. For example, the processor 350 may acquire movement information (movement data) of the electronic device 301 via the first sensor 331, based on the identification that the user is in a sleep state.

In an embodiment, the processor 350 may acquire the magnitude of the movement (also referred to as “strength of movement” or “degree of movement”) of the electronic device 301 via the first sensor 331, based on the identification that the user is in a sleep state.

In an embodiment, the processor 350 may set a period (e.g., time point) for detecting (e.g., measuring) a movement of the electronic device 301 via the first sensor 331, based on the identification that the user is in a sleep state.

In one embodiment, the processor 350 may repeatedly detect a movement of the electronic device 301 via the first sensor 331 at designated time points (e.g., from the time the user starts sleeping to the time the user ends sleeping) while the user is in a sleep state. For example, the processor 350 may control the first sensor 331 to initiate an operation of acquiring a signal for detecting a movement of the electronic device 301 at designated time intervals while the user is in a sleep state.

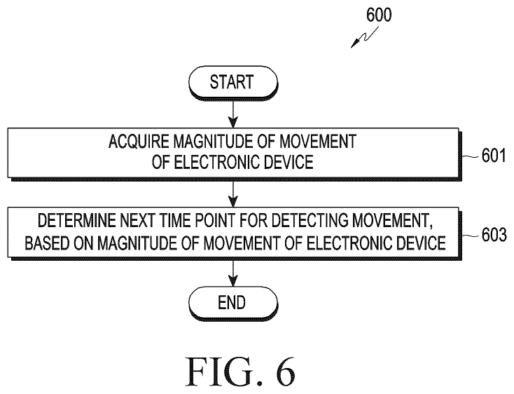

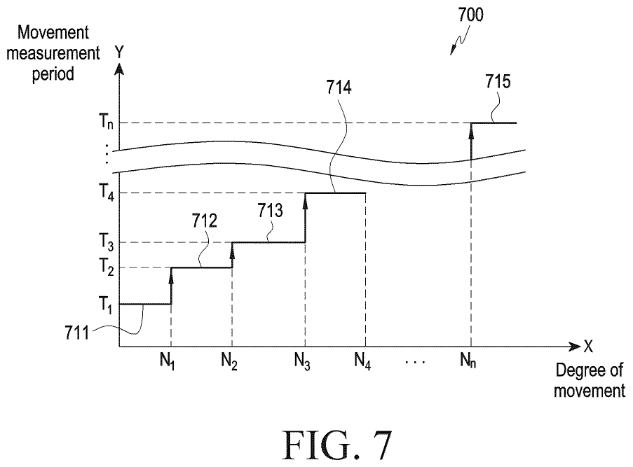

In an embodiment, the processor 350 may variably set (e.g., adjust) a period (or time point) for detecting a movement of the electronic device 301 via the first sensor 331, based on the magnitude of the movement of the electronic device 301 acquired via the first sensor 331 while the user is in a sleep state. For example, the processor 350 may set the next time point at which the movement of the electronic device 301 is to be detected via the first sensor 331, based on the magnitude of the movement of the electronic device 301 acquired via the first sensor 331 at the current time point while the user is in a sleep state. A method for variably setting a period (or time point) for detecting a movement of the electronic device 301, based on the magnitude of the movement of the electronic device 301 acquired via the first sensor 331, will be described in more detail later with reference to FIGS. 6 and 7.

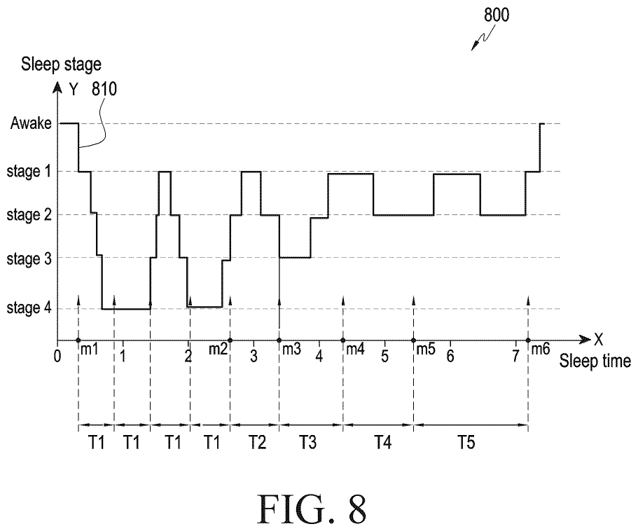

In an embodiment, the processor 350 may variably set a period (or time point) for detecting a movement of the electronic device 301, based on the time elapsed since the user started sleeping. A method for variably setting a period (or time point) for detecting a movement of the electronic device 301, based on the time elapsed since the user has started sleeping will be described in more detail later with reference to FIG. 8.

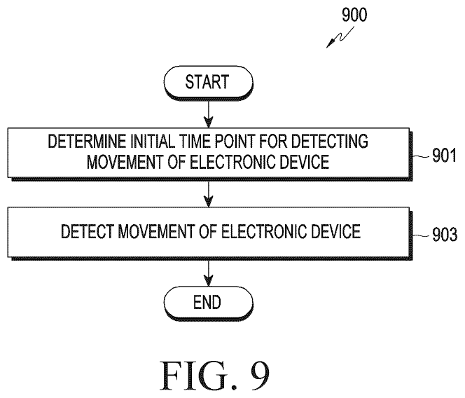

In an embodiment, the processor 350 may determine the initial time point for detecting a movement of the electronic device 301 via the first sensor 331, based on the user's sleep history. A method for determining the initial time point for detecting a movement of the electronic device 301, based on the user's sleep history, will be described in more detail later with reference to FIG. 9.

In an embodiment, the processor 350 may control, based on the identification that the user is in a sleep state, a setting related to an operation of acquiring a time corresponding to a designated sleep stage among sleep stages of the sleep state, which will be described later in operation 407.

In an embodiment, in operation 405, the processor 350 may acquire biometric information based on the biosignal detected via the second sensor 332, based on the magnitude of the movement of the electronic device 301 being less than or equal to a threshold magnitude.

In an embodiment, as described in operation 403, the processor 350 may initiate an operation of detecting a movement of the electronic device 301 at each set time point (or period). Based on identification that the magnitude of the movement of the electronic device 301 is less than or equal to the threshold magnitude for a designated time period from the time point at which the movement detection of the electronic device 301 begins, the processor 350 may acquire biometric information, based on the biosignal detected via the second sensor 332 for a designated time period.

In an embodiment, the processor 350 may identify that the magnitude of the movement of the electronic device 301 is less than or equal to the threshold magnitude for a designated time period from a first time point when measurement of the movement of the electronic device 301 begins via the first sensor 331. The processor 350 may acquire a PPG signal via the PPG sensor at a second time point when the magnitude of the movement of the electronic device 301 is identified to be less than or equal to the threshold magnitude for a designated time period from the first time point or for a designated time period from the second time point. For example, the processor 350 may perform a one-time operation of acquiring a PPG signal via the PPG sensor at the second time point when the magnitude of the movement of the electronic device 301 is identified to be less than or equal to the threshold magnitude for a designated time period from the first time point. For example, the processor 350 may repeatedly perform an operation of acquiring a PPG signal (e.g., acquiring the PPG signal approximately two to three times) via the PPG sensor for a designated time period from the second time point at which the magnitude of the movement of the electronic device 301 is less than or equal to the threshold magnitude for a designated time period from the first time point.

In an embodiment, the processor 350 may acquire blood pressure from the PPG signal by using a designated blood pressure estimation algorithm. For example, the processor 350 may extract a pulse wave from the PPG signal and analyze the waveform of the pulse wave (pulse wave analysis), which varies according to cardiac output (e.g., the volume of blood ejected from the atrium) and total peripheral resistance (e.g., the degree of resistance applied by blood vessels to the ejected blood flow), thereby acquiring (e.g., estimate) blood pressure. However, the method for acquiring blood pressure from the PPG sensor is not limited to the example described above.

In an embodiment, when biometric information (e.g., blood pressure) is acquired, the processor 350 may store, in the memory 340, the time at which the biometric information is acquired. For example, the processor 350 may identify that the magnitude of the movement of the electronic device 301 is less than or equal to a threshold magnitude for a designated time period from the first time point. When biometric information is acquired at the second time point (e.g., a time point after a designated time has elapsed from the first time point) at which the magnitude of the movement of the electronic device 301 is identified to be less than or equal to the threshold magnitude, the processor 350 may store, in the memory 340, the second time point (e.g., a time corresponding to the second time point) at which the biometric information is acquired. For example, when the biometric information is acquired for a designated time period from the second time point, the processor 350 may store (e.g., record), in the memory 340, a time (or time section) including the second time point and the time point after a designated time has elapsed from the second time point.

In an embodiment, the processor 350 may perform the operation of detecting a movement of the electronic device 301 in operation 403 and the operation of acquiring biometric information in operation 405 repeatedly (e.g., at set time points (or periods)) while the user is in a sleep state (e.g., (e.g., from the time the user starts sleeping to the time the user ends sleeping). When biometric information is acquired by repeatedly performing operation 403 and operation 405, the processor 350 may store, in the memory 340, the acquired biometric information along with the time at which the biometric information is acquired, each time biometric information is acquired. Hereinafter, a set of times at which the biometric information is acquired and stored in the memory 340 together with the biometric information will also be referred to as “first time period.”

In an embodiment, the processor 350 may not perform an operation of acquiring biosignals acquired via the second sensor 332, based on the magnitude of the movement of the electronic device 301 exceeding the threshold magnitude.

In an embodiment, in operation 407, the processor 350 may acquire a time period corresponding to a designated sleep stage among sleep stages of the sleep state.

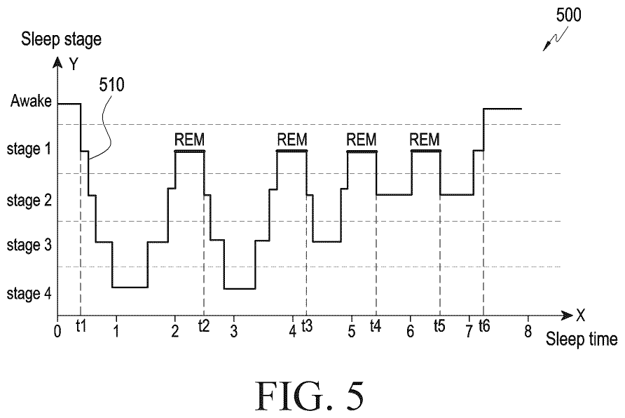

Hereinafter, the sleep stage (and sleep cycle) will be first described with reference to FIG. 5.

FIG. 5 is a diagram 500 for illustrating sleep stages according to an embodiment of the disclosure.

In an embodiment, FIG. 5 may be a graph including lines 510 representing sleep stages (or sleep cycles) that change as sleep time progresses.

Referring to FIG. 5, in an embodiment, the sleep stages may include rapid eye movement (REM) sleep stages and non-REM sleep stages. The non-REM sleep stages may include a first sleep stage (stage 1), a second sleep stage (stage 2), a third sleep stage (stage 3), and a fourth sleep stage (stage 4).

In an embodiment, the sleep stages may be classified based on the ratio (or proportion) of brainwaves (e.g., alpha waves (brainwaves having a frequency range of approximately 8 Hz to approximately 12 Hz), theta waves (brainwaves having a frequency range of approximately 4 Hz to approximately 8 Hz), and/or delta waves (brainwaves having a frequency range of approximately 0.5 Hz to approximately 4 Hz)) measured via the second sensor 332 and including brainwaves having different frequency ranges and/or based on the amplitude (and eye movements) of the measured brainwaves.

In an embodiment, the REM sleep stage may be a sleep stage in which the eyes move rapidly from side to side, brain activity is heightened, dreaming occurs, cerebral blood flow and oxygen consumption increase, and irregular heartrate and breathing rate are observed.

In an embodiment, the first sleep stage (the first sleep stage of the non-REM sleep stage) and the second sleep stage (the second sleep stage of the non-REM sleep stage) may be referred to as “light sleep stages.” In the first sleep stage and the second sleep stage, brainwaves including theta waves (brainwaves having a frequency range of approximately 4 Hz to approximately 8 Hz) and alpha waves (brainwaves having a frequency range of approximately 8 Hz to approximately 12 Hz)) may be measured. The first sleep stage and the second sleep stage may be the sleep stages in which the user is in a sleep state but may be easily awakened by small sounds.

In an embodiment, the third sleep stage (the third sleep stage of the non-REM sleep stage) and the fourth sleep stage (the fourth sleep stage of the non-REM sleep stage) may be referred to “deep sleep stages.” In the third sleep stage and the fourth sleep stage, brainwaves including delta waves (brainwaves having a frequency range of approximately 0.5 Hz to approximately 4 Hz) may be measured. The third sleep stage and the fourth sleep stage may be the sleep stages in which all muscles, except for those required for respiration, are at rest, and sleep state is not easily terminated.

In an embodiment, the third sleep stage and the fourth sleep stage may be the sleep stages in which the user's movement is substantially absent and the biosignals remain constant, and may be stable states suitable for acquiring biometric information.

Referring to FIG. 5, the non-REM sleep stage is illustrated as including the first sleep stage to the fourth sleep stage, but it is not limited thereto. For example, the non-REM sleep stage may include a different number of sleep stages depending on the classification method used for sleep stages.

In an embodiment, the sleep stages may repeat, and when the user sleeps for approximately six hours, three to four sleep cycles may occur. For example, in FIG. 5, a first sleep cycle may have a time period from the time point (t1) at which sleep begins to the time point (t2) at which REM sleep ends, a second sleep cycle may have a time period from the time point (t2) at which the REM sleep ends to the time point (t3) at which the subsequent REM sleep ends, a third sleep cycle may have a time period from the time point (t3) at which the REM sleep ends to the time point (t4) at which the subsequent REM sleep ends, and a fourth sleep cycle may have a time period from the time point (t4) at which the REM sleep ends to the time point (t5) at which the subsequent REM sleep ends. In FIG. 5, the user's sleep may end at time point (t6).

In an embodiment, as illustrated in FIG. 5, the third sleep stage and the fourth sleep stage (deep sleep stage) may be longer in the first sleep cycle of the sleep cycles than in other sleep cycles. In an embodiment, as illustrated in FIG. 5, the third sleep stage and the fourth sleep stage (deep sleep stage) may become shorter as the sleep progresses from the first sleep cycle to the fourth sleep cycle.

Referring back to FIG. 4, in an embodiment, a processor (e.g., the processor 350 of FIG. 3) may acquire (e.g., determine) a sleep stage via a first sensor (e.g., the first sensor 331 of FIG. 3) and/or a second sensor (e.g., the second sensor 332 of FIG. 3). For example, the processor 350 may acquire sleep stages over time during sleep, based on the movement of the electronic device 301 (or the movement of a user wearing the electronic device 301) detected via the first sensor 331 (e.g., an acceleration sensor), and a heart rate variability, a respiration rate, and/or a heart rate acquired via the second sensor 332 (e.g., a PPG sensor, an ECG sensor).

In an embodiment, the processor 350 may acquire sleep stages over time during sleep, based on the user's breathing sound input to the electronic device 301 via a microphone (e.g., a microphone included in the input module 150 of FIG. 1) included in the electronic device 301 while the user is in a sleep state. In an embodiment, the processor 350 may control an external electronic device (e.g., the electronic devices 102, 104 of FIG. 1) such that the external electronic device acquires sleep stages over time during sleep, based on the user's breathing sound while the user is in a sleep state. The processor 350 may acquire sleep stages over time during sleep by receiving, from the external electronic device via a communication module (e.g., the communication module 310 of FIG. 3), the sleep stage acquired by the external electronic device according to the sleep time.

In an embodiment, the processor 350 may acquire sleep stages over time during sleep by cooperating with an external electronic device that communicates with the electronic device 301. For example, the processor 350 may acquire sleep stages over time during sleep by using a Wi-Fi-based technology that measures the user's breathing (and the user's movement) by using a Wi-Fi signal transmitted and received between the electronic device 301 and the external electronic device while the user is positioned between the location of the electronic device 301 and the location of the external electronic device.

However, the method for acquiring sleep stages over time during sleep is not limited to the examples described above.

In an embodiment, the processor 350 may, when sleep stages over time during sleep are acquired using the above-described methods, identify a time period corresponding to a designated sleep stage (e.g., a deep sleep stage) (e.g., a set of time periods during which the deep sleep stage is measured out of the entire time the user is in a sleep state). Hereinafter, a time period corresponding to the designated sleep stage (e.g., a deep sleep stage) will also be referred to as “second time period” or “second time period corresponding to a designated sleep stage.”

In an embodiment, the processor 350 may acquire a second time period corresponding to a designated sleep stage among sleep stages of a sleep state after the user's sleep state ends. For example, the processor 350 may acquire biosignals (and movements of the electronic device 301) for acquiring sleep stages via a sensor module (e.g., the first sensor 331 and/or the second sensor 332) while the user is in a sleep state. After the user's sleep state ends, the processor 350 may acquire sleep stages over time during sleep, based on the acquired biosignals (and movements of the electronic device 301) for acquiring the sleep stages, thereby acquiring the second time period corresponding to the designated sleep stage. For example, the processor 350 may control an external electronic device such that the external electronic device acquires sleep stages over time during sleep while the user is in a sleep state. After the user's sleep state ends, the processor 350 may acquire the second time period corresponding to a designated sleep stage by acquiring sleep stages over time during sleep from the external electronic device via the communication module 310. However, the disclosure is not limited thereto, and the processor 350 may acquire biosignals (and movements of the electronic device 301) for acquiring sleep stages in real time via a sensor module (e.g., the first sensor 331 and/or the second sensor 332) while the user is in a sleep state, and acquire sleep stages over time during sleep, based on the acquired biosignal, thereby acquiring the second time period corresponding to a designated sleep stage.

In an embodiment, when the second time period corresponding to the designated sleep stage is acquired, the processor 350 may store, in memory (the memory 340 of FIG. 3), the acquired second time period together with information on the sleep stage acquired during the second time period.

In an embodiment, in operation 409, the processor 350 may identify, among the acquired biometric information, biometric information acquired during a third time period, the third time period being an overlapping time period between the first time period during which the biometric information is acquired and the second time period corresponding to the designated sleep stage.

In an embodiment, the processor 350 may identify the first time period during which the biometric information is acquired while performing operation 405 and the second time period corresponding to the designated sleep stage acquired through operation 407. The processor 350 may identify the third time period, which is an overlapping time period between the first time period and the second time period (e.g., the time period overlapped with the second time period within the first time period). The processor 350 may identify the biometric information acquired during the third time period, among the biometric information acquired during the first time period (e.g., biometric information acquired through operation 405).

In an embodiment, as described above, the first time period during which the biometric information is acquired may be a set of second time points at which the magnitude of the movement of the electronic device 301 is identified to be less than or equal to a threshold magnitude (e.g., when the biometric information is measured at each of the second time points) or a set of time periods from each second time point to a time point after a designated time period (e.g., when the biometric information is measured from each second time point to a time point after a designated time period). The first time period during which the biometric information is acquired may be a time during which the user is in a physically stable state while sleeping.

In an embodiment, as described above, the second time period corresponding to the designated sleep stage may be a set of time periods during which the deep sleep stage is measured among the entire time the user is in a sleep state. The second time period corresponding to the designated sleep stage may be a time during which the user is in a mentally stable state while sleeping.

In an embodiment, biometric information acquired during the third time period may be identified (e.g., extracted) among the biometric information acquired during the first time period (e.g., blood pressure acquired during the first time period), thereby acquiring biometric information while the user is in a physically and mentally stable state during sleep, the third time period (e.g., blood pressure acquired during the third time period) being an overlapping time period between the first time period during which the biometric information is acquired and the second time period corresponding to the designated sleep stage.

Although not illustrated in FIG. 4, in an embodiment, the processor 350 may identify that the user's sleep state ends, via the first sensor 331 (e.g., acceleration sensor). In an embodiment, operation 409 may be an operation performed after the user's sleep state ends.

In an embodiment, the processor 350 may display, via the display module 320, information related to the biometric information identified through operation 409.

FIG. 6 is a flowchart 600 for illustrating a method for setting a detection time point for a movement of the electronic device 301, based on the magnitude of the movement of the electronic device 301 according to an embodiment of the disclosure.

In the following embodiments, respective operations may be performed sequentially, but are not necessarily performed sequentially. For example, the order of the operations may be changed, and at least two operations may be performed in parallel.

According to an embodiment, operations 601 to 603 may be understood as being performed by a processor (e.g., the processor 350 of FIG. 3) of an electronic device (e.g., the electronic device 301 of FIG. 3).

FIG. 7 is a diagram 700 for illustrating a method for setting a detection time point for a movement of the electronic device 301, based on the magnitude of the movement of the electronic device 301 according to an embodiment of the disclosure.

In an embodiment, the operations of FIG. 6 may be included in an operation of detecting a movement of an electronic device (e.g., the electronic device 301 of FIG. 3) via a first sensor (e.g., the first sensor 331 of FIG. 3) in operation 403 of FIG. 4.

Referring to FIGS. 6 and 7, in operation 601, in an embodiment, a processor (e.g., the processor 350 of FIG. 3) may acquire the magnitude of the movement of the electronic device 301 via the first sensor 331 at a time point set to measure the movement of the electronic device 301 (or for a designated time period from the time point), based on identification that the user is in a sleep state.

In operation 603, in an embodiment, the processor 350 may determine a next time point for detecting (e.g., measuring) the movement of the electronic device 301, based on the magnitude of the movement of the electronic device 301. For example, when the magnitude of the movement of the electronic device 301 is acquired at a first time point (e.g., a current time point), the processor 350 may determine a second time point (e.g., the time point immediately following the current time point at which the movement of the electronic device 301 is to be detected), based on the acquired magnitude of the movement of the electronic device 301. For example, when the magnitude of the movement of the electronic device 301 is acquired for a designated time period from the first time point (e.g., the current time point), the processor 350 may acquire (e.g., calculate) an average of the magnitudes of the movements of the electronic device 301 acquired for the designated time period. The processor 350 may determine a second time point at which the movement of the electronic device 301 is to be measured (e.g., the time point immediately following the current time point at which the motion of the electronic device 301 is to be measured), based on the acquired average of the magnitudes of the movements of the electronic device 301.

In an embodiment, as the magnitude of the movement becomes greater, the processor 350 may determine the next time point for detecting a movement of the electronic device 301 such that the time from the current time point at which the movement of electronic device 301 is detected to the next time point for detecting a movement of electronic device 301 becomes relatively longer. As the magnitude of the movement of electronic device 301 becomes smaller, the processor 350 may determine the next time point for detecting a movement of the electronic device 301 such that the time from the current time point at which the movement of electronic device 301 is detected to the next time point becomes relatively shorter.

In an embodiment, the processor 350 may determine the next time point for detecting a movement of the electronic device 301, based on a movement range corresponding to the magnitude of the movement of the electronic device 301.

In an embodiment, the processor 350 may set a plurality of movement ranges. For example, as illustrated in FIG. 7, the processor 350 may set a plurality of movement ranges including a first movement range (e.g., a range in which the magnitude of the movement is equal to or greater than 0 and less than N1), a second movement range (e.g., a range in which the magnitude of the movement (also referred to as the “degree of movement) is equal to or greater than N1 and less than N2), a third movement range (e.g., a range in which the magnitude the movement is equal to or greater than N2 and less than N3), a fourth movement range (e.g., a range in which the magnitude the movement is equal to or greater than N3 and less than N4, and an nth movement range (here, n is a natural number equal to or greater than 5) (e.g., a range in which the magnitude the movement is equal to or greater than Nₙ₋₁ to less than Nₙ). As the range progresses from the first movement range to the nth movement range, the representative value (e.g., median, mode, average, minimum, or maximum) of each movement range may become larger.

In an embodiment, as the movement range progresses from the first movement range to the nth movement range, the processor 350 may determine the next time point for detecting a movement of the electronic device 301 such that the time from the current time point at which the movement is detected to the next time point (also referred to as a “movement detection period”) for detecting a movement of electronic device 301 becomes relatively longer. For example, when the magnitude of the movement of electronic device 301 corresponds to the first movement range (e.g., when the magnitude of the movement of the electronic device 301 falls within a range equal to or greater than 0 and less than N1), the processor 350 may determine the movement detection period as T1, as illustrated by line 711. When the magnitude of the movement corresponds to the second movement range (e.g., when the magnitude of the movement of the electronic device 301 falls within a range of equal to or greater than N1 and less than N2), the processor 350 may determine the movement detection period as T2, as illustrated by line 712. When the magnitude of the movement corresponds to the third movement range (e.g., when the magnitude of the movement of the electronic device 301 falls within a range of equal to or greater than N2 and less than N3), the processor 350 may determine the movement detection period as T3, as illustrated by line 713. When the magnitude of the movement corresponds to the fourth movement range (e.g., when the magnitude of the movement of the electronic device 301 falls within a range of equal to or greater than N3 and less than N4), the processor 350 may determine the movement detection period as T4, as illustrated by line 714. When the magnitude of the movement corresponds to the nth movement range (e.g., when the magnitude of the movement of the electronic device 301 falls within a range of equal to or greater than Nn−1 and less than Nn), the processor 350 may determine the movement detection period as Tn, as illustrated by line 715.

In an embodiment, the operation in which the processor 350 sets a plurality of movement ranges and movement measurement periods corresponding to the plurality of movement ranges may be performed prior to operation 401 in FIG. 4, which identifies whether the user is in a sleep state.

In an embodiment, as the magnitude of the movement of the electronic device 301 becomes smaller, the movement detection period may be determined to be relatively shorter, thereby acquiring biometric information more frequently when the magnitude of the movement is smaller during the user's sleep state. In an embodiment, as the magnitude of the movement of the electronic device 301 becomes larger, the movement detection period may be determined to be relatively longer, thereby enabling reduction in power consumption while the electronic device 301 performs operations for providing biometric information.

FIG. 8 is a diagram 800 for illustrating a method for setting a period for detecting a movement of the electronic device 301, based on the elapsed time from the time point at which sleep begins, according to an embodiment of the disclosure.

In an embodiment, the operation for setting a period for detecting a movement of the electronic device 301, based on the elapsed time from the time point at which sleep begins, may be performed prior to operation 401 in FIG. 4, which identifies whether the user is in a sleep state.

Referring to FIG. 8, in an embodiment, FIG. 8 may be a graph including a line 810 representing sleep stages over time during sleep.

In an embodiment, a processor (e.g., the processor 350 of FIG. 3) may set a period for detecting a movement of the electronic device 301 such that, as more time elapses from the time point at which sleep begins (e.g., time point (m1)) (e.g., as time elapses from the time point at which sleep begins), the period for detecting a movement of the electronic device 301 becomes relatively longer.

In an embodiment, the processor 350 may set a period for detecting a movement of the electronic device 301, based on a time range corresponding to the elapsed time elapsed from the time point at which sleep begins.

In an embodiment, the processor 350 may set a plurality of time ranges, based on the elapsed time from the time point at which sleep begins. For example, in FIG. 8, the processor 350 may set a plurality of time ranges including a first time range (e.g., a time range from time point (m1) to time point (m2)), a second time range (e.g., a time range from time point (m2) to time point (m3)), a third time range (e.g., a time range from time point (m3) to time point (m4)), a fourth time range (e.g., a time range from time point (m4) to time point (m5)), and a fifth time range (e.g., a time range from time point (m5) to time point (m6)).

In an embodiment, the processor 350 may set different periods for detecting a movement of the electronic device 301 for each of the plurality of time ranges.

In an embodiment, the processor 350 may set a period for detecting a movement of the electronic device 301 such that, the earlier the start time of a time range, the period for detecting movement is set to be relatively shorter within that time range. For example, in the first time range, the processor 350 may set the period for detecting a movement of the electronic device (e.g., the electronic device 301 of FIGS. 3) to T1 and may set, in the second time range, the period for detecting a movement of the electronic device 301 to T2, which is longer than T1. In the third time range, the processor 350 may set the period for detecting a movement of the electronic device 301 to T3, which is longer than T2. In the fourth time range, the processor 350 may set the period for detecting a movement of the electronic device 301 to T4, which is longer than T3. In the fifth time range, the processor 350 may set the period for detecting a movement of the electronic device 301 to T5, which is longer than T4. However, the disclosure is not limited thereto. For example, the processor 350 may set the period for detecting a movement of the electronic device 301 such that the period for detecting a movement of the electronic device 301 in the time ranges corresponding to the third sleep stage and the fourth sleep stage (deep sleep stage) (e.g., time ranges predicted to be deep sleep stage) is shorter than the period for detecting a movement of the electronic device 301 in the time ranges corresponding to sleep stages other than the third sleep stage and the fourth sleep stage (e.g., time ranges predicted to be the first sleep stage, the second sleep stage, and the REM sleep stage).

In an embodiment, as described through FIG. 5, the third sleep stage and the fourth sleep stage (deep sleep stage) may be longer than other sleep cycles in the first sleep cycle of the sleep cycle. In an embodiment, as illustrated in FIG. 5, the third sleep stage and the fourth sleep stage (deep sleep stage) may become shorter as the sleep progresses from the first to the fourth sleep cycle.

In an embodiment, the operation in which the processor 350 sets a plurality of time ranges and movement measurement periods corresponding to the plurality of time ranges may be performed prior to operation 401 in FIG. 4, which identifies whether the user is in a sleep state.

In an embodiment, after the plurality of time ranges and the movement measurement periods corresponding to the plurality of time ranges are set, the processor 350 may perform an operation of detecting a movement of the electronic device 301 (e.g., operation 403 in FIG. 4), based on the set plurality of time ranges and movement measurement periods corresponding to the plurality of time ranges. For example, when it is identified that the user is in a sleep state, the processor 350 may identify a time range corresponding to the elapsed time from the time point at which sleep begins (e.g., the time range to which the elapsed time from the time point at which sleep begins belongs). The processor 350 may detect a movement of the electronic device 301, based on the period for detecting a movement of the electronic device 301 set for the identified time range.

In an embodiment, as time elapses from the time point at which sleep begins, the processor 350 may increase the period for detecting a movement of the electronic device 301, thereby reducing power consumption during the operation in which the electronic device 301 provides biometric information, and enabling more efficient acquisition of biometric information.

In an embodiment, the third and fourth sleep stages (deep sleep stages) may be concentrated within approximately 3 to 4 hours from the time point at which sleep begins. In consideration of this, the period for detecting a movement of the electronic device 301 may be set accordingly. For example, the processor 350 may increase the period for detecting a movement of the electronic device 301 as time elapses from the time point at which sleep begins and may stop detecting a movement of the electronic device 301 after a designated time period (e.g., approximately 3 to 4 hours) has elapsed from the time point at which sleep begins. For example, the processor 350 may set the period for detecting a movement of the electronic device 301 to be relatively longer as time elapses from the time point at which sleep begins, and after a designated time period (e.g., approximately 3 to 4 hours) has elapsed from the time point at which sleep begins, may detect a movement of the electronic device 301 at a period that is longer than other periods set to detect a movement of the electronic device 301 before the designated time period.

FIG. 9 is a flowchart 900 for illustrating a method for determining an initial time point for detecting a movement of the electronic device 301, based on a user's sleep history, according to an embodiment of the disclosure.

In the following embodiments, the respective operations may be performed sequentially, but are not necessarily performed sequentially. For example, the order of the operations may be changed, and at least two operations may be performed in parallel.

According to an embodiment, operations 901 to 903 may be understood as being performed by a processor (e.g., the processor 350 of FIG. 3) of an electronic device (e.g., the electronic device 301 of FIG. 3).

In operation 901, in an embodiment, the processor 350 may determine an initial time point for detecting a movement of the electronic device 301, based on the user's sleep history.

In an embodiment, the processor 350 may determine the time period from the time point at which the user starts sleeping to the time point at which the user enters a designated sleep stage (e.g., a deep sleep stage) by analyzing a history of sleep stages according to previously acquired sleep time. For example, the processor 350 may analyze previously performed operations for providing biometric information, thereby acquiring the average time taken by the user to enter the third sleep stage of the non-REM stage (e.g., to the time point at which the third sleep stage begins) after the user starts sleeping. The processor 350 may set an initial time point for detecting a movement of the electronic device 301 such that the movement of the electronic device 301 is detected via the first sensor (e.g., the first sensor 331 of FIG. 3) at the time point at which the acquired average time has elapsed from the time point at which the user starts sleeping. In an embodiment, the operation in which the processor 350 analyzes previously performed operations for providing biometric information and acquires the average time taken by the user to enter the third sleep stage of the non-REM stage after the user starts sleeping may be performed prior to operation 401 of identifying whether the user is in a sleep state.

In operation 903, in an embodiment, the processor 350 may detect a movement of the electronic device 301 via the first sensor 331, based on the time point determined through operation 901. For example, after the user starts sleeping, the processor 350 may start an operation of detecting a movement of the electronic device 301 via the first sensor 331 at the time point determined through operation 901.

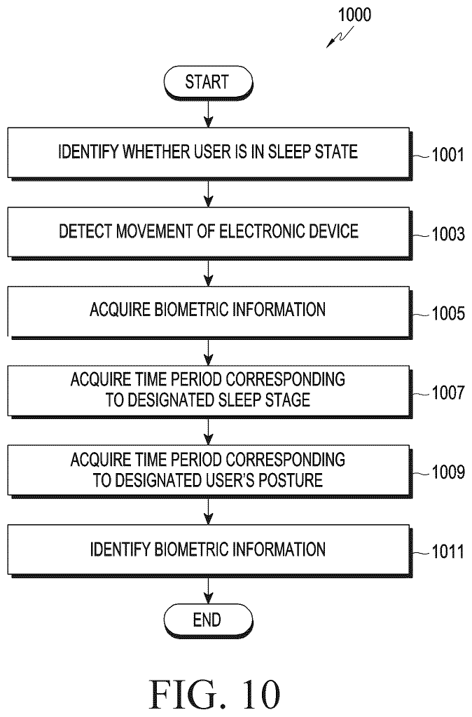

FIG. 10 is a flowchart 1000 for illustrating a method for providing biometric information according to an embodiment of the disclosure.

In the following embodiments, the respective operations may be performed sequentially, but are not necessarily performed sequentially. For example, the order of the operations may be changed, and at least two operations may be performed in parallel.

According to an embodiment, operations 1001 to 1011 may be understood as being performed by a processor (e.g., the processor 350 of FIG. 3) of an electronic device (e.g., the electronic device 301 of FIG. 3).

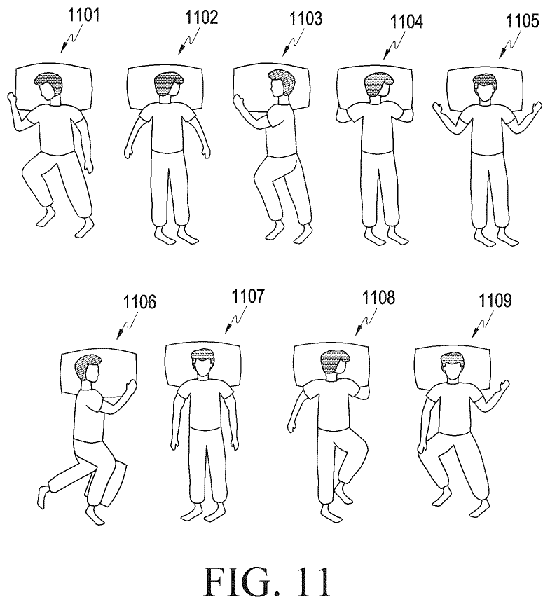

FIG. 11 illustrates a method for providing biometric information according to an embodiment of the disclosure.

Referring to FIGS. 10 and 11, in operation 1001, in an embodiment, the processor 350 may identify whether the user is in a sleep state.

Since operation 1001 is at least partially identical or similar to operation 401 in FIG. 4, a detailed description thereof will be omitted.

In operation 1003, in an embodiment, the processor 350 may detect a movement of the electronic device 301 via the first sensor (e.g., the first sensor 331 of FIG. 3), based on the identification that the user is in a sleep state.

Since operation 1003 is at least partially identical or similar to operation 403 in FIG. 4, a detailed description thereof will be omitted.

In operation 1005, in an embodiment, the processor 350 may acquire biometric information based on a biosignal acquired via the second sensor (e.g., the second sensor 332 of FIG. 3), based on the magnitude of the movement of the electronic device 301 being less than or equal to a threshold magnitude.

Since operation 1005 is at least partially identical or similar to operation 405 in FIG. 4, a detailed description thereof will be omitted.

In operation 1007, in an embodiment, the processor 350 may acquire a time period corresponding to a designated sleep stage among sleep stages of the sleep state.

Since operation 1007 is at least partially identical or similar to operation 407 in FIG. 4, a detailed description thereof will be omitted.

In operation 1009, in an embodiment, the processor 350 may acquire a time period corresponding to a designated user posture, based on information acquired via the first sensor 331.

In an embodiment, the user may change posture while in a sleep state. For example, during sleep, the user may unconsciously (or consciously) assume various postures. For example, as shown by reference numerals 1101, 1102, 1103, 1104, 1105, 1106, 1107, 1108, and 1109 in FIG. 11, the user may unconsciously assume various postures during sleep.

In an embodiment, depending on the user's posture during sleep, the posture (e.g., the position of the wrist relative to other parts of the user's body) of the user's part (e.g., the wrist that is the target for acquiring biometric information by the electronic device 301) wearing the electronic device 301 may vary.

In an embodiment, when the user is in a posture in which the body part wearing the electronic device 301 is compressed by other parts of the user's body (e.g., the torso, the head) and biometric information (e.g., blood pressure) is being acquired by the electronic device 301, accurate biometric information may not be acquired. For example, when an operation for providing biometric information during sleep is performed while the electronic device 301 is worn on the user's right wrist, accurate blood pressure may not be acquired in case that the user is in a posture in which the right wrist (e.g., the wrist wearing the electronic device) is compressed by other parts of the user's body, as shown in reference numerals 1103, 1104, and 1108.

In an embodiment, the processor 350 may acquire a posture (e.g., position and direction) of the electronic device 301 (and/or a change in the posture of the electronic device 301) during sleep, via the first sensor 331 (e.g., an acceleration sensor). The processor 350 may acquire a posture of the user over time during sleep, based on the acquired posture of the electronic device 301. From the posture of the user over time during sleep, the processor 350 may identify a time period (hereinafter, also referred to as a “fourth time period”) during which the user is in a designated user's posture (e.g., a posture in which the user's right wrist is not compressed by another part of the user's body) during sleep.

In operation 1011, in an embodiment, the processor 350 may identify, among the biometric information acquired through operation 1005, the biometric information acquired during the third time period, the third time period being an overlapping time period among the first time period during which the biometric information is acquired, the second time period corresponding to the designated sleep stage, and the fourth time period during which the user is in the designated user's posture during sleep.

In one embodiment, the processor 350 may identify, among the biometric information acquired through operation 1005, the biometric information acquired during a time period, which is an overlapping time period among the first time period during which the biometric information is acquired, the second time period corresponding to the designated sleep stage, and the fourth time period during which the user is in the designated user's posture during sleep, thereby acquiring biometric information in a state where the user is physically and mentally stable and the user's biometric information is measurable more accurately.

In an embodiment, the above-described example has described, but is not limited to, identifying, among the biometric information acquired through operation 1005, the biometric information acquired during a time period, which is an overlapping time period among the first time period during which the biometric information is acquired, the second time period corresponding to the designated sleep stage, and the fourth time period during which the user is in the designated user's posture during sleep. For example, in operation 1009, the processor 350 may identify the time period during which the biometric information is acquired in a posture other than the designated user's posture (e.g., a posture in which the user's right wrist is compressed by another part of the user's body, as shown by reference numeral 1103, reference numeral 1104, or reference numeral 1108). The processor 350 may acquire, among the biometric information acquired through operation 1005, biometric information acquired during the third time period, the third time period being an overlapping time period between the first time period during which the biometric information is acquired and the second time period corresponding to the designated sleep stage. The processor 350 may identify, among the biometric information acquired during the third time period, biometric information that is acquired during the portion of the third time period excluding the time period during which the user is in a posture other than the designated posture.

In an embodiment, the processor 350 may display, via a display module (e.g., the display module 320 of FIG. 3), information related to biometric information identified through operation 1011.

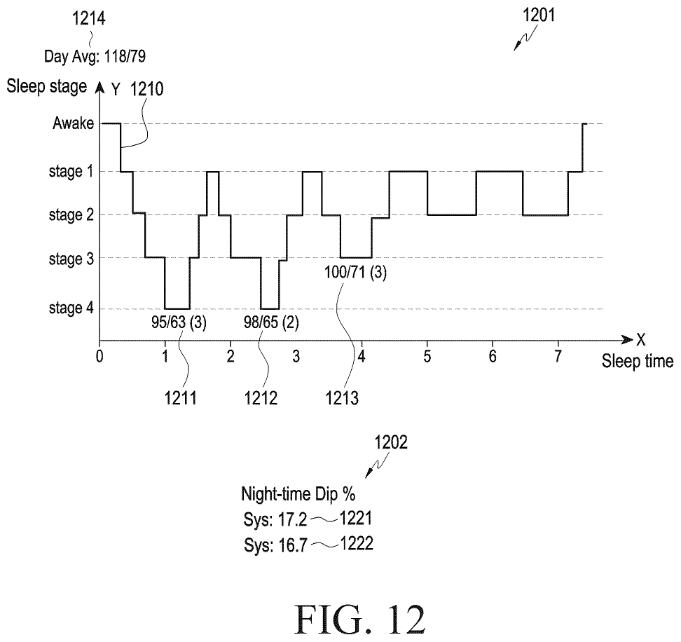

FIG. 12 is a diagram for illustrating a method of providing biometric information according to an embodiment of the disclosure.

Referring to FIG. 12, in an embodiment, a processor (e.g., the processor 350 of FIG. 3) may provide information related to biometric information (e.g., the biometric information identified through operation 409 in FIG. 4 or operation 1011 in

FIG. 10).

In an embodiment, the processor 350 may display, via a display module (e.g., the display module 320 of FIG. 3), biometric information (e.g., blood pressure) acquired during sleep and biometric information acquired during the daytime. For example, as shown by reference numeral 1201 in FIG. 12, the processor 350 may display, via the display module 320, biometric information (e.g., blood pressure) acquired during sleep and biometric information acquired during the daytime on a graph including a line 1210 that represents sleep stages over time during sleep. In FIG. 12, the information 1211 may represent the average systolic blood pressure (e.g., “95” in “95/63(3)”), average diastolic blood pressure (e.g., “63” in “95/63(3)”), and the number of blood pressure measurements (e.g., “3” in “95/63(3)”) acquired during the first sleep cycle. In FIG. 12, the information 1212 may represent the average systolic blood pressure (e.g., “98” in “98/65(2)”), average diastolic blood pressure (e.g., “65” in “98/65(2)”), and the number of blood pressure measurements (e.g., “2” in “98/65(2)”) acquired during the second sleep cycle. In FIG. 12, the information 1213 may represent the average systolic blood pressure (e.g., “100” in “100/71(3)”), average diastolic blood pressure (e.g., “71” in “100/71(3)”), and the number of blood pressure measurements (e.g., “3” in “100/71(3)”) acquired during the third sleep cycle. In FIG. 12, the information 1214 may represent the average systolic blood pressure (e.g., “118” in “Day Avg 118/79”) and the average diastolic blood pressure (e.g., “79” in “Day Avg 118/79”) of the daytime blood pressure.

In an embodiment, the processor 350 may display, via the display module 320, information comparing biometric information (e.g., blood pressure) acquired during sleep with biometric information acquired during non-sleep periods. For example, as shown by reference numeral 1202, the processor 350 may display, via the display module 320, information including the reduction ratio of the average nighttime systolic blood pressure compared to the average daytime systolic blood pressure (e.g., 1221), the reduction ratio of the average nighttime diastolic blood pressure compared to the average daytime diastolic blood pressure (e.g., 1222), and a blood pressure pattern (e.g., dipper pattern, non-dipper pattern, extreme dipper pattern, or riser pattern).

However, the method for providing information related to biometric information is not limited to the examples described with reference to FIG. 12. For example, the processor 350 may compare biometric information (e.g., blood pressure) acquired during sleep, excluding biometric information acquired while the user is in a posture other than the designated posture, with biometric information acquired during non-sleep periods. In addition, the processor 350 may display, via the display module 320, information related to postures other than the designated posture (e.g., the postures illustrated by reference numerals 1103, 1104, and 1108 in FIG. 11). For example, the processor 350 may provide information on potential diseases such as heart disease, stroke, heart failure, and retinal disorders, based on a blood pressure pattern acquired via the comparison of daytime blood pressure and nighttime blood pressure, or may may provide information guiding the timing of taking medication for a user taking antihypertensive medication, such as in the morning when the daytime blood pressure is higher than the nighttime blood pressure, or before bed when the nighttime blood pressure is higher than the daytime blood pressure.

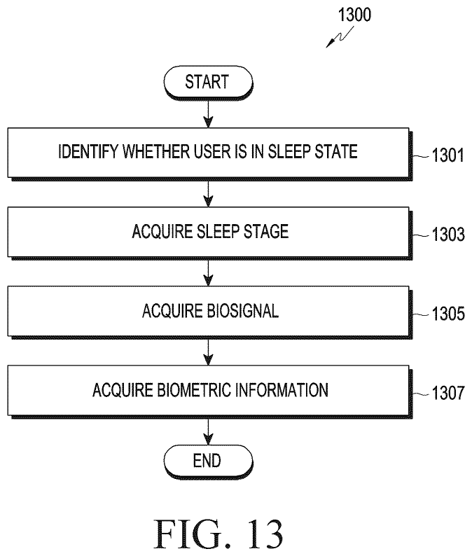

FIG. 13 is a flowchart 1300 for illustrating a method for providing biometric information according to an embodiment of the disclosure.

In the following embodiment, the respective operations may be performed sequentially, but are not necessarily performed sequentially. For example, the order of operations may be changed, and at least two operations may be performed in parallel.

According to an embodiment, operations 1301 to 1307 may be understood as being performed by a processor (e.g., the processor 350 of FIG. 3) of an electronic device (e.g., the electronic device 301 of FIG. 3).

Referring to FIG. 13, in operation 1301, in an embodiment, the processor 350 may identify whether the user is in a sleep state.

Since operation 1301 is at least partially identical or similar to operation 401 of FIG. 4, a detailed description thereof will be omitted.

In operation 1303, in an embodiment, the processor 350 may acquire a sleep stage of the user's sleep state, based on identification that the user is in a sleep state. For example, the processor 350 may perform an operation of acquiring (e.g., monitoring) a sleep stage of the user's sleep state in real time, based on identification that the user is in a sleep state.

In an embodiment, the processor 305 may acquire (e.g., determine) a sleep stage in real time via a first sensor (e.g., the first sensor 331 of FIG. 3) and/or a second sensor (e.g., the second sensor 332 of FIG. 3). For example, the processor 350 may acquire sleep stages in real time over time during sleep, based on the movement of the electronic device 301 (or the movement of the user wearing the electronic device 301) acquired via the first sensor 331 (e.g., an acceleration sensor), and a heart rate variability, a respiration rate, and/or a heart rate acquired via the second sensor 332 (e.g., a PPG sensor, an ECG sensor).

In an embodiment, the processor 350 may acquire sleep stages in real time over time during sleep, based on the user's breathing sound input to the electronic device 301 via a microphone (e.g., a microphone included in the input module 150 of FIG. 1) included in the electronic device 301 while the user is in a sleep state. In an embodiment, the processor 350 may control an external electronic device such that the external electronic device acquires sleep stages in real time over time during sleep, based on the user's breathing sound while the user is in a sleep state. The processor 350 may acquire sleep stages over time during sleep by receiving in real time, from the external electronic device via a communication module (e.g., the communication module 310 of FIG. 3), the sleep stage acquired by the external electronic device according to the sleep time.

In an embodiment, the processor 350 may acquire sleep stages in real time over time during sleep by cooperating with an external electronic device that communicates with the electronic device 301. For example, the processor 350 may acquire sleep stages in real time over time during sleep by using a Wi-Fi-based technology that measures the user's breathing (and the user's movement) by using a Wi-Fi signal transmitted and received between the electronic device 301 and the external electronic device while the user is positioned between the location of the electronic device 301 and the location of the external electronic device.

In operation 1305, in an embodiment, the processor 350 may acquire a biosignal (e.g., a PPG signal) via the second sensor 332, based on the identification that the user's sleep stage is a designated sleep stage (e.g., a deep sleep stage).

In operation 1307, in an embodiment, the processor 350 may acquire biometric information (e.g., blood pressure), based on the biosignal acquired in operation 1305.

In an embodiment, when biometric information is acquired (or while acquiring biometric information), the processor 350 may identify the time period during which the biometric information is acquired, and store the identified information in memory (e.g., the memory 340 of FIG. 3).

In an embodiment, the processor 350 may acquire the user's biometric information during sleep by repeatedly performing operations 1303 to 1307 from the time point at which the user's sleep state is identified until the time point at which the user's sleep state ends. In an embodiment, the processor 350 may acquire information related to the user's biometric information during sleep (e.g., average blood pressure during sleep), based on the acquired biometric information and the time period during which the biometric information is acquired.

In an embodiment, during a designated sleep stage (e.g., a deep sleep stage), the magnitude of the user's movement may be less than or equal to the magnitude of the designated movement (e.g., the magnitude of the user's movement at which accurate blood pressure measurement is possible). As described above, the designated sleep stage may correspond to a sleep stage in which the user is in a mentally stable state. Accordingly, biometric information measured during the designated sleep stage may be biometric information measured while the user is in both a physically and mentally stable state.

FIG. 13 illustrates an example in which biosignals (and biometric information) are acquired when it is identified that the user is in a designated sleep stage, but it is not limited thereto. For example, the processor 350 may acquire biometric information at a time point at or during a time period, when the user is in a designated sleep stage and the magnitude of the movement of the electronic device 301 acquired via the first sensor 331 is less than or equal to a threshold magnitude.

In an embodiment, although not illustrated in FIG. 13, the processor 350 may further perform the operations described through FIGS. 6 to 12 while performing the operations of FIG. 13.

Referring to FIGS. 4 to 13, blood pressure is used as an example of the biometric information provided by the electronic device 301 (e.g., the biometric information identified in operation 409 or operation 1011), but it is not limited thereto. For example, the biometric information that may be provided by the electronic device 301 may include not only blood pressure, but also biometric information, such as heart rate, which may be accurately measured when the user is in a physically and mentally stable state.

An electronic device 301 according to an embodiment may include a first sensor 331 configured to detect a movement of the electronic device 301, a second sensor 332 configured to measure a biosignal of a user wearing the electronic device 301, at least one processor 350 operably connected to the first sensor 331 and the second sensor 332, and memory 340 storing instructions. The instructions, when executed by the at least one processor 350, may cause the electronic device 301 to detect a movement of the electronic device 301 via the first sensor 331, based on identification that the user is in a sleep state. The instructions, when executed by the at least one processor 350, may cause the electronic device 301 to acquire biometric information of the user based on a biosignal acquired via the second sensor 332, based on the magnitude of the movement of the electronic device 301 being less than or equal to a threshold magnitude. The instructions, when executed by the at least one processor 350, may cause the electronic device 301 to acquire a time period corresponding to a designated sleep stage among sleep stages of the sleep state. The instructions, when executed by the at least one processor 350, may cause the electronic device 301 to identify, among the acquired biometric information, biometric information acquired during a third time period, the third time period being an overlapping time period between a first time period during which the biometric information is acquired and a second time period corresponding to the designated sleep stage.

In an embodiment, the instructions, when executed by the at least one processor 350, may cause the electronic device 301 to acquire biometric information of the user based on the biosignal acquired via the second sensor 332, based on the magnitude of the movement of the electronic device 301 being less than or equal to a threshold magnitude for a designated time period from a time point at which the movement of the electronic device 301 is detected.

In an embodiment, the instructions, when executed by the at least one processor 350, may cause the electronic device 301 to determine a next time point for detecting a movement of the electronic device 301, based on the magnitude of the movement of the electronic device 301 acquired for the designated time period from the time point at which the movement of the electronic device 301 is detected.

In an embodiment, the instructions, when executed by the at least one processor 350, may cause the electronic device 301 to, after a plurality of time ranges and movement detection periods corresponding to the plurality of time ranges are set, identify a time range corresponding to an elapsed time from a time point at which the user starts sleeping, among the plurality of time ranges, and to detect a movement of the electronic device 301 via the first sensor 331, based on the movement detection period corresponding to the identified time range.

In an embodiment, the instructions, when executed by the at least one processor 350, may cause the electronic device 301 to determine an initial time point for detecting a movement of the electronic device 301, based on a sleep history of the user.

In an embodiment, the designated sleep stage may include a third sleep stage and a fourth sleep stage of a non-rapid eye movement (REM) sleep stage.

In an embodiment, the instructions, when executed by the at least one processor 350, may cause the electronic device 301 to acquire a fourth time period corresponding to a designated posture of the user while the user is in a sleep state, and to identify, among the acquired biometric information, biometric information acquired during a fifth time period, the fifth time period being an overlapping time period among the first time period during which the biometric information is acquired, the second time period corresponding to the designated sleep stage, and the fourth time period corresponding to the designated posture of the user.

In an embodiment, the electronic device 301 may further include a display module 320, and the instructions, when executed by the at least one processor 350, may cause the electronic device 301 to identify a pattern of the biometric information by comparing the identified biometric information with biometric information acquired during the non-sleep state of the user, and to display information related to the identified pattern via the display module 320.

In an embodiment, the biometric information may include blood pressure and/or heart rate.

A method for providing biometric information by an electronic device 301 according to an embodiment may include detecting a movement of the electronic device 301 via a first sensor 331 of the electronic device 301, based on identification that a user of the electronic device 301 is in a sleep state. The method may include acquiring biometric information of the user based on a biosignal acquired via a second sensor 332 of the electronic device 301, based on the magnitude of the movement of the electronic device 301 being less than or equal to a threshold magnitude. The method may include acquiring a time period corresponding to a designated sleep stage among sleep stages of the sleep state. The method may include identifying, among the acquired biometric information, biometric information acquired during a third time period, which is an overlapping time period between a first time period during which the biometric information is acquired and a second time period corresponding to the designated sleep stage.

In an embodiment, the acquiring of the biometric information of the user may include acquiring biometric information of the user based on the biosignal acquired via the second sensor 332, based on the magnitude of the movement of the electronic device 301 being less than or equal to a threshold magnitude for a designated time period from a time point at which the movement of the electronic device 301 is detected.

In an embodiment, the detecting of the movement of the electronic device 301 may include determining a next time point for detecting a movement of the electronic device 301, based on the magnitude of the movement of the electronic device 301 acquired for the designated time period from the time point at which the movement of the electronic device 301 is detected.

In an embodiment, the detecting of the movement of the electronic device 301 may include, identifying, after a plurality of time ranges and movement detection periods corresponding to the plurality of time ranges are set, a time range corresponding to an elapsed time from a time point at which the user starts sleeping, among the plurality of time ranges, and detecting a movement of the electronic device 301 via the first sensor 331, based on the movement detection cycle corresponding to the identified time range.

In an embodiment, the detecting of the movement of the electronic device 301 may include determining an initial time point for detecting a movement of the electronic device 301, based on a sleep history of the user.

In an embodiment, the designated sleep stage may include a third sleep stage and a fourth sleep stage of a non-rapid eye movement (REM) sleep stage.

In an embodiment, the method may further include acquiring a fourth time period corresponding to a designated posture of the user while the user is in a sleep state, and identifying, among the acquired biometric information, biometric information acquired during a fifth time period, the fifth time period being an overlapping time period among the first time period during which the biometric information is acquired, the second time period corresponding to the designated sleep stage, and the fourth time period corresponding to the designated posture of the user.

In an embodiment, the method may further include identifying a pattern of the biometric information by comparing the identified biometric information with biometric information acquired during the non-sleep state of the user, and displaying information related to the identified pattern via the display module 320 of the electronic device 301.

In an embodiment, the biometric information may include blood pressure and/or heart rate.

An electronic device 301 according to an embodiment may include a sensor configured to measure a biosignal of a user wearing the electronic device 301 and at least one processor 350 operatively connected to the sensor. The at least one processor 350 may be configured, based on identification that the user is in a sleep state, to acquire a sleep stage of the sleep state. The at least one processor 350 may be configured to acquire the biosignal via the sensor, based on identification that the sleep stage is a designated sleep stage. The at least one processor 350 may be configured to acquire biometric information of the user based on the biosignal.

In an embodiment, the designated sleep stage may include a third sleep stage and a fourth sleep stage of a non-REM sleep stage.

In an embodiment, a non-transitory computer-readable medium storing computer-executable instructions, wherein the computer-executable instructions, when executed by at least one processor 350 of an electronic device 301, may cause the electronic device 301 to detect a movement of the electronic device 301 via a first sensor 331 of the electronic device 301, based on identification that a user of the electronic device 301 is in a sleep state. The computer-executable instructions, when executed by the at least one processor 350 of the electronic device 301, may cause the electronic device 301 to acquire biometric information of the user based on a biosignal acquired via a second sensor 332 of the electronic device 301, based on the magnitude of the movement of the electronic device 301 being less than or equal to a threshold magnitude. The computer-executable instructions, when executed by the at least one processor 350 of the electronic device 301, may cause the electronic device 301 to acquire a time period corresponding to a designated sleep stage among sleep stages of the sleep state. The computer-executable instructions, when executed by the at least one processor 350 of the electronic device 301, may cause the electronic device 301 to identify, among the acquired biometric information, biometric information acquired during a third time period, the third time period being an overlapping time period between a first time period during which the biometric information is acquired and a second time period corresponding to the designated sleep stage.

In an embodiment, the computer-executable instructions, when executed by the at least one processor 350 of the electronic device 301, may cause the electronic device 301 to acquire biometric information of the user based on the biosignal acquired via the second sensor 332, based on the magnitude of the movement of the electronic device 301 being less than or equal to a threshold magnitude for a designated time period from the time point at which the movement of the electronic device 301 is detected.

In addition, the structure of the data used in the embodiments of the disclosure described above may be recorded on a computer-readable recording medium through various means. The computer-readable recording medium includes storage media such as magnetic storage media (e.g., read only memory (ROM), floppy disk, hard disk, etc.) and optical reading media (e.g., CD-ROM, digital versatile disc (DVD), etc.).

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device comprising:
a first sensor configured to detect a movement of the electronic device;
a second sensor configured to measure a biosignal of a user wearing the electronic device;
at least one processor including processing circuitry; and
memory storing instructions that, when executed by the at least one processor individually or collectively, cause the electronic device to:
based on identifying that the user is in a sleep state, detect the movement of the electronic device via the first sensor,
based on a magnitude of the movement of the electronic device being less than or equal to a threshold magnitude, acquire, based on the biosignal acquired via the second sensor, biometric information of the user,
acquire a time period corresponding to a designated sleep stage among sleep stages of the sleep state, and
identify, among the acquired biometric information, biometric information acquired during a third time period, the third time period being an overlapping time period between a first time period during which the biometric information is acquired and a second time period corresponding to the designated sleep stage.

2. The electronic device of claim 1, wherein the instructions, when executed by the at least one processor individually or collectively, cause the electronic device to:
based on the magnitude of the movement of the electronic device being less than or equal to the threshold magnitude for a designated time period from a time point at which the movement of the electronic device is detected, acquire, based on the biosignal acquired via the second sensor, the biometric information of the user.

3. The electronic device of claim 2, wherein the instructions, when executed by the at least one processor individually or collectively, cause the electronic device to:
based on the magnitude of the movement of the electronic device acquired for the designated time period from the time point at which the movement of the electronic device is detected, determine a next time point for detecting the movement of the electronic device.

4. The electronic device of claim 2, wherein the instructions, when executed by the at least one processor individually or collectively, cause the electronic device to:
after a plurality of time ranges and movement detection periods respectively corresponding to the plurality of time ranges are set, identify, among the plurality of time ranges, a time range corresponding to an elapsed time from a time point at which the user starts sleeping, and
based on a movement detection period corresponding to the identified time range, detect the movement of the electronic device via the first sensor.

5. The electronic device of claim 1, wherein the instructions, when executed by the at least one processor individually or collectively, cause the electronic device to:
based on a sleep history of the user, determine an initial time point for detecting the movement of the electronic device.

6. The electronic device of claim 1, wherein the designated sleep stage comprises a third sleep stage and a fourth sleep stage of a non-rapid eye movement (REM) sleep stage.

7. The electronic device of claim 1, wherein the instructions, when executed by the at least one processor individually or collectively, further cause the electronic device to:
acquire a fourth time period corresponding to a designated posture of the user while the user is in a sleep state, and
identify, among the acquired biometric information, biometric information acquired during a fifth time period, the fifth time period being an overlapping time period among the first time period during which the biometric information is acquired, the second time period corresponding to the designated sleep stage, and the fourth time period corresponding to the designated posture of the user.

8. The electronic device of claim 1, further comprising:
a display,
wherein the instructions, when executed by the at least one processor individually or collectively, further cause the electronic device to:
identify a pattern of the biometric information by comparing the identified biometric information with biometric information acquired during a non-sleep state of the user, and
display information related to the identified pattern via the display.

9. The electronic device of claim 1, wherein the biometric information comprises at least one of blood pressure or heart rate.

10. A method performed by an electronic device, the method comprising:
based on identifying that a user wearing the electronic device is in a sleep state, detecting a movement of the electronic device via a first sensor of the electronic device;
based on a magnitude of the movement of the electronic device being less than or equal to a threshold magnitude, acquiring, based on a biosignal of the user acquired via a second sensor of the electronic device, biometric information of the user;
acquiring a time period corresponding to a designated sleep stage among sleep stages of the sleep state; and
identifying, among the acquired biometric information, biometric information acquired during a third time period, the third time period being an overlapping time period between a first time period during which the biometric information is acquired and a second time period corresponding to the designated sleep stage.

11. The method of claim 10, wherein the acquiring of the biometric information of the user comprises:
based on the magnitude of the movement of the electronic device being less than or equal to the threshold magnitude for a designated time period from a time point at which the movement of the electronic device is detected, acquiring, based on the biosignal acquired via the second sensor, biometric information of the user.

12. The method of claim 11, wherein the detecting of the movement of the electronic device comprises:
based on the magnitude of the movement of the electronic device acquired for the designated time period from the time point at which the movement of the electronic device is detected, determining a next time point for detecting the movement of the electronic device.

13. The method of claim 11, wherein the detecting of the movement of the electronic device comprises:
after a plurality of time ranges and movement detection periods respectively corresponding to the plurality of time ranges are set, identifying, among the plurality of time ranges, a time range corresponding to an elapsed time from a time point at which the user starts sleeping; and
based on a movement detection period corresponding to the identified time range, detecting the movement of the electronic device via the first sensor.

14. The method of claim 10, wherein the detecting of the movement of the electronic device comprises:
based on a sleep history of the user, determining an initial time point for detecting the movement of the electronic device.

15. The method of claim 10, wherein the designated sleep stage comprises a third sleep stage and a fourth sleep stage of a non-rapid eye movement (REM) sleep stage.

16. The method of claim 10, further comprising:
acquiring a fourth time period corresponding to a designated posture of the user while the user is in a sleep state; and
identifying, among the acquired biometric information, biometric information acquired during a fifth time period, the fifth time period being an overlapping time period among the first time period during which the biometric information is acquired, the second time period corresponding to the designated sleep stage, and the fourth time period corresponding to the designated posture of the user.

17. The method of claim 10, further comprising:
identifying a pattern of the biometric information by comparing the identified biometric information with biometric information acquired during a non-sleep state of the user; and
displaying, via a display of the electronic device, information related to the identified pattern via the display.

18. The method of claim 10, wherein the biometric information comprises at least one of blood pressure or heart rate.

19. One or more non-transitory computer-readable storage media storing instructions that, when executed by at least one processor of an electronic device individually or collectively, cause the electronic device to perform operations, the operations comprising:
based on identifying that a user wearing the electronic device is in a sleep state, detecting a movement of the electronic device via a first sensor of the electronic device;
based on a magnitude of the movement of the electronic device being less than or equal to a threshold magnitude, acquiring, based on a biosignal of the user acquired via a second sensor of the electronic device, biometric information of the user;
acquiring a time period corresponding to a designated sleep stage among sleep stages of the sleep state; and
identifying, among the acquired biometric information, biometric information acquired during a third time period, the third time period being an overlapping time period between a first time period during which the biometric information is acquired and a second time period corresponding to the designated sleep stage.

20. The one or more non-transitory computer-readable storage media of claim 19, the operations further comprising:
based on the magnitude of the movement of the electronic device being less than or equal to the threshold magnitude for a designated time period from a time point at which the movement of the electronic device is detected, acquiring, based on the biosignal acquired via the second sensor, biometric information of the user.
